(19) United States
(12) Patent Application Publication
(10) Pub. No.: US 2015/0241424 A1
(43) Pub. Date: Aug. 27, 2015

(54) MULTI-ANALYTE ASSAY

(71) Applicants: Gregory M. LAWRENCE, (US); Lisa SHINEFELD, (US); VERAX BIOMEDICAL INCORPORATED, Marlborough, MA (US)

(72) Inventors: Gregory M. Lawrence, West Boylston, MA (US); Lisa Shinefeld, Lexington, MA (US)

(21) Appl. No.: 14/433,370

(22) PCT Filed: Oct. 7, 2013
(86) PCT No.: PCT/US13/63749

§ 371 Date: Apr. 2, 2015

Related U.S. Application Data
(60) Provisional application No. 61/772,523, filed on Mar. 4, 2013.
(60) Provisional application No. 61/710,651, filed on Oct. 5, 2012.

(51) Int. Cl. G01N 33/543; G01N 33/569; G01N 33/553

(52) U.S. Cl. CPC G01N 33/54353; G01N 33/553; G01N 33/56911; G01N 2405/04; G01N 2400/50

(57) ABSTRACT

The present invention is directed to devices and methods using pan-generic antibodies to detect bacteria in a sample.

### CROSS-REFERENCE TO RELATED APPLICATIONS

This application claims the benefit of U.S. Provisional Patent Application Nos. 61/710,651 and 61/772,523, filed Oct. 5, 2012 and Mar. 4, 2013, respectively. The disclosure of each of those applications is incorporated by reference herein in its entirety.

### FIELD OF THE INVENTION

The invention relates to binding assays, especially immunoassays, utilizing a multivalent binding agent immobilized on a particle. The invention also relates to the surprising discovery that increasing the size of the particles improves the sensitivity of the screen.

### BACKGROUND OF THE INVENTION

Testing liquid samples for bacterial contamination is a critical component in a wide variety of fields, such as medicine (e.g., testing blood samples for transfusion), environmental safety (e.g., testing water samples for human use), and food safety (e.g., testing food and beverage samples for consumption). The importance of bacterial testing necessitates tests that are rapid, sensitive, and broadly specific enough to detect a wide variety of bacterial species and genera. Practical limitations, such as the amount of a detection reagent (e.g., a bacterial antigen-binding antibody) or the visibility of a “positive” result in an assay may control the ability of current bacterial testing methods to meet these requirements. Thus, there is a need for improved reagents, devices and methods for rapidly, broadly and sensitively detecting bacteria.

### BRIEF SUMMARY OF THE INVENTION

In a first aspect, the invention provides a lateral flow device for detecting bacteria in a sample, the device comprising a flow path for the sample and further comprising a pan-generic binding agent specific for one or more bacterial antigens, wherein the pan-generic binding agent is immobilized on a population of particularly-sized detectable particles; and a capture binding agent that captures the population of particles bound to bacterial antigens, wherein the capture binding agent is immobilized on the flow path, and wherein the population of detectable particles are disposed along the flow path such that the sample contacts the population of detectable particles before contacting the capture binding agent. In some embodiments, the detectable particle is a chemiluminescent, a luminescent, a fluorescent, a magnetic or a colored particle. For convenience, the term “colored particle” will be used but the invention contemplates embodiments using other forms of detectable particles. In embodiments utilizing a colored particle, the particle may be a gold, silver, or platinum particle. In some embodiments, the particle is from about 60 to about 120 nm in diameter. In some embodiments the particle is about 80 nm in diameter.

In some embodiments, the pan-generic binding agent specifically binds a Gram-positive bacterial antigen. In some such embodiments, the pan-generic polyclonal antibody binds lipoteichoic acid (LTA). In some embodiments, the pan-generic binding agent specifically binds a Gram-negative bacterial antigen. In some such embodiments, the pan-generic polyclonal antibody binds a bacterial lipopolysaccharide structure (LPS). In some embodiments, at least one pan-generic binding agent specifically binds a Gram-positive bacterial antigen and at least one pan-generic binding agent specifically binds a Gram-negative bacterial antigen. In some embodiments, the pan-generic binding agent is capable of binding three or more genera of bacteria. In some embodiments, the pan-generic binding agent is immobilized on the detectable particle via a linker. In some embodiments, the linker is protein A, protein G, or protein L.

In some embodiments, the pan-generic binding agent is an antibody. In some embodiments the pan-generic binding agent comprises two or more pan-generic antibodies, wherein each pan-generic antibody specifically binds one or more bacterial antigens. In various embodiments, each pan-generic antibody is immobilized on a separate subpopulation or on the same subpopulation of colored particles. According to the invention, at least one pan-generic antibody is immobilized on a population of particularly sized colored particles. In some embodiments, the pan-generic binding agent can be combined with one or more binding agents that is not pan-generic. For example, a binding agent that is not pan-generic may bind one or more species or strains of bacteria but not to multiple genera.

In some embodiments, the pan-generic antibody is selected from a polyclonal antibody, a monoclonal antibody and a combination of polyclonal and monoclonal antibodies. In some embodiments, the pan-generic antibody is polyclonal and binds a plurality of bacterial antigens. In some embodiments, the pan-generic antibody is polyclonal and binds a plurality of Gram-positive bacterial antigens. In some embodiments, the pan-generic antibody is polyclonal and binds a plurality of Gram-negative bacterial antigens. In some embodiments, the pan-generic antibody is polyclonal and binds a plurality of Gram-negative bacterial antigens and Gram-positive bacterial antigens. In some embodiments, at least one pan-generic antibody is a monoclonal pan-generic antibody and at least one pan-generic antibody is a polyclonal pan-generic antibody.

In some embodiments, the pan-generic antibody specifically binds a Gram-positive bacterial antigen. In some embodiments, the pan-generic antibody specifically binds a Gram-negative bacterial antigen. In some embodiments, at least one pan-generic antibody specifically binds a Gram-positive bacterial antigen and at least one pan-generic antibody specifically binds a Gram-negative bacterial antigen. In some embodiments, the pan-generic antibody is capable of binding three or more genera of bacteria. In some embodiments, the pan-generic binding antibody is immobilized on the colored particle via a linker.

In some embodiments, the device comprises at least three pan-generic binding agents that specifically bind Gram-positive bacterial antigens, each pan-generic binding agent immobilized on a separate subpopulation of colored particles; and at least three pan-generic binding agents that specifically bind Gram-negative bacterial antigens, each pan-generic binding agent immobilized on a separate subpopulation of colored particles. In some embodiments, at least one pan-generic binding agent is an antibody. In some embodiments, at least one pan-generic antibody is a monoclonal antibody. In some embodiments, the subpopulations of particles are of different sizes. In some embodiments, the particles are gold, silver, or platinum. In some embodiments, at least some of the particles are from about 60 nm to about 120 nm in diameter. In some embodiments, at least some of the particles are gold particles from about 60 nm to about 120 nm in diameter. In some embodiments, at least one particle population (e.g., a gold particle population) comprises a 80 nm particle. In some embodiments, at least one particle population (e.g., a gold particle population) comprises a 40 nm particle.

In some embodiments, the capture binding agent is a pan-generic antibody that specifically binds a bacterial antigen. In some embodiments, the capture binding agent is the same as the pan-generic binding agent. In some embodiments, the capture antibody is immobilized in one or more locations on the sample flow path. In some embodiments, the sample flow path is an absorbent membrane. In some embodiments, the absorbent membrane is nitrocellulose.

In some embodiments the colored particles are dried within a solid support surface disposed above the absorbent membrane and in contact with the upper surface of the membrane.

In a second aspect, the invention provides a method for detecting the presence or absence of bacteria in a sample, comprising contacting the sample with a pan-generic binding agent specific for a bacterial antigen, wherein the pan-generic binding agent is immobilized on an particularly-sized colored particle, and wherein the sample is contacted with the pan-generic binding agent under conditions that permit binding between the pan-generic binding agent and a bacterial antigen to form a binding agent-bacterial antigen complex, and further comprising contacting an immobilized capture binding agent specific to a bacterial antigen with the particularly-sized colored particles under conditions that permit binding between the immobilized capture binding agent and the particle-pan-generic binding agent-bacterial antigen complex, wherein capture of the colored particle with the pan-generic binding agent by the capture binding agent indicates the presence of bacteria in the sample. In some embodiments, a small amount of soluble pan-generic binding agent is added to the sample before the assay is performed. Such small amount is an amount sufficient to improve the signal of the system.

In some embodiments, the method comprises contacting a device according to the first aspect of the invention with a sample under conditions that permit binding of the capture antibody to the colored particle with the pan-generic antibody, wherein capture of the particle by the capture antibody indicates the presence of bacteria in the sample.

In some embodiments, the sample has been pre-treated.

According to the invention, a sample can be any liquid sample that is suspected of containing bacteria. In some embodiments, the sample is a biological fluid, including urine, sputum, spinal fluid, ascites, blood and blood products. In some embodiments, the sample is any liquid sample that is suspected of containing bacteria. In some embodiments, the sample is blood or a blood product. In some embodiments the blood or blood product is selected from the group consisting of: whole blood, leukocytes, hematopoietic stem cells, platelets, red blood cells, plasma, bone marrow and serum.

In some embodiments, the blood or a blood product such as platelets is from a donor for transfusion to a recipient. In some embodiments the sample is a dialysis sample. In some embodiments, the dialysis sample is selected from hemodialysis fluid and peritoneal dialysis fluid. In some embodiments, the sample is a sample of fluid in which a tissue such as a tissue from a donor for transplanting to a recipient has been stored. In some embodiments, the tissue is selected from the group consisting of: blood cell cultures, stem cell cultures, skin and bone and cartilage graft materials. In some embodiments, the sample is a sample from lung, bronchoalvealor, peritoneal, or arthroscopic lavage. In some embodiments, the samples are environmental samples such as water and soil. In some embodiments, the samples are foods or beverages. Those of skill in the art will recognize that in cases where the sample source is in solid form, such as soil or solid foods, the sample may be liquid that is extracted from the solid form or liquid that has been in contact with the solid form. In some embodiments, the sample is a biological sample, for example, urine, tears, sputum or cerebrospinal fluid.

In a third aspect, the invention provides a reagent for use in a binding assay comprising a particle selected from a gold particle, a silver particle and a platinum particle, wherein the particle size is from about 60 nm to about 120 nm, and wherein the particle is bound to a multi-specific pan-generic binding agent. In some embodiments, the particle size is about 80 nm. In some embodiments, the pan-generic binding agent is bound to the particle via a linker. In some embodiments, the linker is selected from protein A, protein G and protein L. In some embodiments, the linker is protein A. In some embodiments, the particle is gold.

In a fourth aspect, the invention provides a method for detecting a substance in a sample comprising mixing the sample with a reagent according to the third aspect of the invention, wherein binding of the substance to the reagent creates a detectable complex; and detecting the complex.

### BRIEF DESCRIPTION OF THE DRAWINGS

FIG. 1A is a graph illustrating that the use of larger colloidal gold particles results in higher signal intensity on the capture line at various numbers of particles per reaction. FIG. 1B is a photograph from a 50% dilution series of 40 nm (“current”) gold particles in a model lateral flow system where staphylococcal protein A coated particles were captured on rabbit IgG capture lines. FIG. 1C is a set of photographs from a 50% dilution series of 80 nm (“enhanced”) gold particles in the model lateral flow system where staphylococcal protein A coated particles were captured on rabbit IgG capture lines.

FIG. 2 is a photograph taken from model lateral flow strips. These results were generated from tenfold dilutions of 8 different bacterial lysates and were derived starting from a 10⁸ stock solution, and the resulting samples were processed in lateral flow strips.

### DETAILED DESCRIPTION OF THE PREFERRED EMBODIMENTS

Unless otherwise defined herein, scientific and technical terms used in this application shall have the meaning commonly understood by those skilled in the art. The techniques and procedures described or referenced herein are well understood and commonly employed using conventional methodologies that are well known and commonly used in the art.

All publications, patents and published patent applications referred to in this application are specifically incorporated by reference herein.

Each embodiment of the invention described herein may be taken alone or in combination with one or more other embodiments of the invention.

### DEFINITIONS

Unless specified otherwise, the following definitions are provided for specific terms, which are used in the above written description.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

As used herein, “a particularly sized particle” is used to mean a particle that provides greater signal in a multianalyte system than a similarly prepared 40 nm particle. In various embodiments, the particles may be a detectable particle such as a chemiluminescent, a luminescent, a fluorescent, a magnetic or a colored particle. In embodiments using a colored particle, the particle can be selected from gold, silver, and platinum particles. In various embodiments, the particularly-sized particles may be, about 60 nm to about 120 nm, including about 60, about 70, about 80, about 90, about 100, about 110 or about 120 nm. In some embodiments, a particularly-sized particle may be 80-100 nm. In some embodiments, this particularly-sized particle is about 80 nm.

As used herein, a “linker” is any chemical moiety that is bound to a particle and to a binding agent, including without limitation, proteins, other biomolecules and other organic chemical compounds.

As used herein, a “multivalent binding agent” is a mixture of binding agents that specifically bind substances in a multianalyte sample, i.e., that comprise multiple specificities. One example of a multivalent binding agent is a polyclonal antibody that can bind more than one antigen of a bacterium and, thus, is multivalent.

As used herein, a “multianalyte sample” is a sample containing multiple substances having binding properties different from each other i.e., a sample that contains a plurality of different binding targets. By way of non-limiting example, a multianalyte sample may be a sample containing a plurality of different bacteria or a plurality of different proteins.

As used herein, “specifically binds” means that a pan-generic antibody recognizes and binds to a particular antigen or set of antigens (e.g., a polypeptide, carbohydrate, lipid, or glycoprotein), but does not bind non-specifically to other molecules in a sample. Likewise, an antigen bound by a pan-generic antibody that specifically binds that antigen is said to be “specifically bound” by that pan-generic antibody. Preferably, a pan-generic antibody that specifically binds a ligand forms an association with that ligand with an affinity of at least 10⁶ M⁻¹, more preferably, at least 10⁷ M⁻¹, even more preferably, at least 10⁸ M⁻¹, and most preferably, at least 10⁹ M⁻¹ either in water, under physiological conditions, or under conditions which approximate physiological conditions with respect to ionic strength, e.g., 140 mM NaCl, and pH, e.g., 7.2.

As used herein, a “pan-generic” binding agent is a binding agent that binds more than one genus of bacteria. Pan-generic binding agents are capable of detecting more than one genus of bacteria when used in the devices and methods of the invention, for example, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more genera of bacteria. In some embodiments, the pan-generic binding agent is one or more pan-generic antibody, as described for the first aspect. In some embodiments, a pan-generic binding agent specifically binds an antigen present in more than one genus of bacteria. By way of non-limiting example, an antibody that specifically binds lipopolysaccharide on two or more genera of Gram-negative bacteria is a pan-generic binding agent. Likewise, an antibody that specifically binds lipoteichoic acid (LTA) on two or more genera of Gram-positive bacteria is a pan-generic binding agent. Such pan-generic binding agents can be polyclonal or monoclonal. In some embodiments, a pan-generic binding agent comprises antibodies with different specificities in a mixture, such that the mixture binds more than one genus of bacteria. Other non-antibody molecules may serve as pan-generic binding agents if they have the capability of binding to bacterial components (e.g. antibiotics such as polymyxin bind to lipopolysaccharides of multiple genera of Gram-negative bacteria, and vancomycin can bind to components of the cell wall of Gram-positive bacteria). These molecules, with a suitable linker, could be used as pan-generic binding agents.

As used herein, “antigen” (for example, a Gram-negative bacterial antigen or a Gram-positive bacterial antigen) is used to mean any molecule, in any structural conformation which may be specifically bound by a pan-generic binding agent. The site on the antigen which is bound by the pan-generic binding agent is called a “binding site.” An antigen may be, without limitation, a protein, a glycoprotein, a carbohydrate, or a lipid.

As used herein, “Gram-positive bacteria” means a strain, type, species, or genera of bacteria that, when exposed to the Gram stain, retains the dye and is, thus, stained blue-purple.

As used herein, “Gram-negative bacteria” means a strain, type, species, or genera of bacteria that, when exposed to the Gram stain, does not retain the dye and is not stained blue-purple. The skilled practitioner will recognize that depending on the concentration of the dye and on the length of exposure, a Gram-negative bacterium may pick up a slight amount of Gram stain and become stained light blue-purple. However, in comparison to a Gram-positive bacterium stained with the same formulation of Gram stain for the same amount of time, a Gram-negative bacterium will be much lighter blue-purple in comparison to a Gram-positive bacterium.

As used herein, “blood or blood product” includes any cell found in blood or bone marrow, as well as any product derived from the blood or bone marrow including, without limitation, whole blood, red blood cells, platelets, serum, plasma, hematopoietic stem cells, and leukocytes (including lymphocytes). The ordinarily skilled biologist will understand that without addition of anti-clotting agents such as EDTA or heparin, whole blood will clot, rendering the majority of the blood cells unusable in transfusion. Accordingly, included in the term, “blood or blood product,” is blood treated with any anti-clotting agent. In addition, during the isolation of particular blood products (e.g., platelets using platelet pheresis), non-blood components, such as physiological saline may be added to the blood. Accordingly, also included in the term, “blood or blood product,” is blood to which has been added any biologically inert substance, such as physiological saline, water, or a storage nutrient solution.

### Devices

In one aspect, the invention provides a device for detecting bacteria in a sample, the device comprising a flow path for the sample and further comprising a pan-generic antibody wherein the pan-generic antibody is specific for one or more bacterial antigens, and wherein the pan-generic antibody is immobilized on a population of particles, and a capture antibody that captures the population of particles that are bound to a bacterial antigen, wherein the capture antibody is immobilized on the flow path, and wherein the population of particles are disposed along the flow path such that the sample contacts the population of particles before contacting the capture antibody. In some embodiments, the particle is a colored particle.

In certain embodiments, the device comprises two or more pan-generic antibodies, wherein each pan-generic antibody is specific for one or more bacterial antigens. In some embodiments, each pan-generic antibody is immobilized on a separate subpopulation of particles. In some embodiments, the particle is a colored particle. In some embodiments, the particle is a colored gold particle.

In certain embodiments, the pan-generic antibody is immobilized on the particle via a linker. In some embodiments, the linker is protein A, protein G, or protein L. In embodiments wherein the device or method comprises two or more pan-generic antibodies, at least one of the pan-generic antibodies is immobilized on the particle via a linker. In some embodiments, the particle is a colored particle.

In certain embodiments, the pan-generic antibody is a polyclonal antibody, monoclonal antibody, or a combination thereof. The pan-generic antibody may specifically bind a Gram-positive bacterial antigen or a Gram-negative bacterial antigen or a combination of Gram-positive and Gram-negative bacterial antigens. In certain embodiments, the device or method of the invention comprises at least one pan-generic antibody that specifically binds a Gram-positive bacterial antigen and at least one pan-generic antibody that specifically binds a Gram-negative bacterial antigen. In certain embodiments, the device or method of the invention comprises at least one, at least two, at least three, at least four, at least five, at least six, at least seven, at least eight, at least nine, or at least ten pan-generic antibodies that bind to a Gram-positive bacterial antigen. In certain embodiments, the device or method of the invention comprises at least one, at least two, at least three, at least four, at least five, at least six, at least seven, at least eight, at least nine, or at least ten pan-generic antibodies that bind to a Gram-negative bacterial antigen. In certain embodiments, the device or method of the invention comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty pan-generic antibodies, wherein the pan-generic antibodies are a mix of pan-generic antibodies that bind to a Gram-positive bacterial antigen and pan-generic antibodies that bind to a Gram-negative bacterial antigen. In some embodiments, antibodies that bind a Gram-negative bacterial antigen are immobilized on a separate subpopulation of particles than antibodies that bind Gram-positive bacterial antigens so that the presence or absence of Gram-negative and Gram-positive bacteria can be detected separately.

A pan-generic binding agent may comprise one or more polyclonal antibodies wherein the polyclonal antibodies are directed against one antigen or multiple antigens. A pan-generic binding agent may comprise one or more monoclonal antibodies or a combination of polyclonal and monoclonal antibodies. In some embodiments, a polyclonal antibody and monoclonal antibodies are immobilized on separate subpopulations of particles. In embodiments comprising a plurality of monoclonal antibodies with different specificities, each specificity may be immobilized on a separate subpopulation of particles. In embodiments comprising multiple polyclonal antibodies with different specificities, each specificity may be immobilized on a separate subpopulation of particles. In some embodiments, the subpopulations of particles are different sizes, colors or both.

In some embodiments, a capture binding agent is a polyclonal antibody, monoclonal antibody, or a combination thereof. In certain embodiments, a capture antibody is a pan-generic antibody that specifically binds a bacterial antigen bound by the pan-generic antibody immobilized on a particle. In certain embodiments, a capture antibody is the same as a pan-generic antibody immobilized on a particle.

In some embodiments, the invention provides a device that is a lateral flow device. In some embodiments, the invention provides a device comprising one or more absorbent membranes. Those of skill in the art will be familiar with materials suitable for use as an absorbent membrane in such devices. In certain embodiments, the absorbent membrane is a nitrocellulose membrane. In some embodiments, the invention provides a device comprising a flow path on which one or more capture antibodies are immobilized. In certain embodiments, two or more, three or more, four or more, five or more, six or more, seven or more, eight or more, nine or more, or ten or more capture antibodies are immobilized on the flow path. In certain embodiments, the capture antibodies are immobilized in one or more locations on the flow path. In specific embodiments, the capture antibodies are immobilized in two or more, three or more, four or more, five or more, six or more, seven or more, eight or more, nine or more, or ten or more locations on the flow path. In some embodiments, each of the one or more locations comprises the same capture antibody. In some embodiments, each of the one or more locations comprises different capture antibodies.

In some embodiments, the invention provides a device comprising pan-generic antibody that is immobilized on a population of detectable particles via a linker, wherein the particles are dried within a support surface disposed above an absorbent membrane and in contact with the upper surface of the membrane where the area of contact between the support surface and the absorbent membrane controls the rate of reconstitution of the particles and/or the time between reconstitution and contacting a capture antibody. In some embodiments, the detectable particle is a chemiluminescent, a luminescent, a fluorescent, a magnetic or a colored particle. In embodiments utilizing a colored particle, the particle may be a gold, silver, or platinum particle. In some embodiments, the particle is from about 60 to about 120 nm in diameter. In some embodiments the particle is about 80 nm in diameter.

In some embodiments, the device comprises a positive control. In some embodiments, the device comprises a location on a flow path indicating that the sample has flowed past the capture antibodies.

In certain embodiments, such a pan-generic binding agent comprises an antibody which binds under physiological conditions to an antigen-containing epitope of a lipopolysaccharide (LPS) structure of a Gram-negative bacteria or a lipoteichoic acid (LTA) structure of a Gram-positive bacteria.

Pan-generic antibodies useful in the devices and methods of the invention include a monoclonal antibody, a polyclonal antibody, a single-chain antibody, a synthetic antibody, a recombinant antibody, a chimeric antibody, or any antigen-binding fragment of the above, including, but not limited to, F(ab), F(ab′), F(ab′)₂, scFv fragments and recombinant fragments. The pan-generic antibodies may be from non-species, for example, a chicken antibody, or from a mammalian species, including but not limited to rabbits, rodents (including mice, rats and guinea pigs), goats, pigs, sheep, camels and humans. The pan-generic antibodies also may be humanized or chimeric antibodies.

Those skilled in the art are enabled to make any such antibody derivatives using standard art-recognized techniques. For example, Jones et al. (Nature 321: 522-525 (1986)) discloses replacing the CDRs of a human antibody with those from a mouse antibody. Marx (Science 229: 455-456 (1985)) discusses chimeric antibodies having mouse variable regions and human constant regions. Rodwell (Nature 342: 99-100 (1989)) discusses lower molecular weight recognition elements derived from antibody CDR information. Clackson (Br. J. Rheumatol. 3052: 36-39 (1991)) discusses genetically engineered monoclonal antibodies, including Fv fragment derivatives, single chain antibodies, fusion proteins chimeric antibodies and humanized rodent antibodies. Reichman et al. (Nature 332: 323-327 (1988)) discloses a human antibody on which rat hypervariable regions have been grafted. Verhoeyen et al. (Science 239: 1534-1536 (1988)) teaches grafting of a mouse antigen binding site onto a human antibody.

Most preferably, the pan-generic antibodies of the present invention are polyclonal antibodies or monoclonal antibodies. Generation of monoclonal and polyclonal antibodies is well within the knowledge of one of ordinary skill in the art of biology (see, e.g., Ausubel et al., Current Protocols in Molecular Biology, John Wiley & Sons, New York, N.Y., 1994). A number of procedures are useful in producing antibodies to the desired unique target antigens. Traditional immunization and harvesting techniques will result in the creation of polyclonal antibodies directed against the common determinants of the target bacterial species including pan-generic determinants such as LPS and LTA. Additionally, cellular hybridization techniques can be utilized to produce immortal hybridoma cell lines that generate specific monoclonal antibodies to the target species.

Antibodies having potential utility for broadly detecting Gram-positive bacteria include those described in Fisher et al., PCT Publication No. WO98/57994; Jackson, D. E. et al., Infection and Immunity 43: 800 (1984); Hamada, S. et al, Microbiol. Immunol. 28: 1009 (1984); Aasjord, P. et al., Acta Path. Microbiol. Immunol. Scand. Sect. C, 93: 245 (1985); McDaniel, L. S. et al., Microbial Pathogenesis 3: 249 (1987); Tadler, M. B. et al., Journal of Clinical Laboratory Analysis 3: 21 (1989); and Stuertz, K et al., Journal of Clinical Microbiology 36: 2346 (1998).

Antibodies having potential utility for broadly detecting Gram-negative bacteria include those described in Nelles, M. J. et al, Infect. Immun. 46: 677 (1984); Teng, N. N. H. et al, Proc. Natl. Acad. Sci. USA 82: 1790 (1985); Dunn, D. L. et al., Surgery 98: 283 (1985); De Jongh-Leuvenink, J. et al, Eur. J. Clin. Microbiol. 5: 148 (1986); Bogard, W. C. et al., Infect. Immun. 55: 899 (1987); Pollack, M. et al., Bacterial Endotoxins: Pathophysiological Effects, Clinical Significance, and Pharmacological Control. pp. 327-338 Alan R. Liss, Inc. (1988); Priest, B. P. et al., Surgery 106: 147 (1989); Tyler, J. W. et al., Journal of Immunological Methods 129: 221 (1990); Siegel, S. A. et al., Infect. Immun. 61: 512 (1993); Shelburne, C. E. et al., J. Periodont. Res. 28: 1 (1993); Di Pardova, F. E. et al., Infect. Immun. 61: 3863 (1993); and De Kievit, T. R. and Lam, J. S. J. Bacteriol. 176: 7129 (1994).

The selection as to which antibody or antibodies to use can be accomplished through classical techniques. Antibody specificity, binding extent and kinetics can be characterized by empirically testing each antibody in an empirical format. Micro-titer screening formats are well documented in the literature to aid in characterizing specific antibody response in any given immunoassay format. Likewise, the activities of detectably labeled antibodies can be characterized by executing a variety of chemical conjugation techniques and screening the resulting product for the optimal performance parameters. The capture antibody and detectably labeled antibody can be screened against the clinical isolates of bacteria from retained platelet or red cell samples to emulate final assay performance as close to final product embodiment as possible. This experimentation leads to the selection and optimization of antibody reagents for application in the various assay formats described below.

Monoclonal antibodies with specificity towards cross-genus targets on the bacterial cell surfaces may be utilized in devices and methods of the invention. In some embodiments, blends of monoclonal antibodies may be utilized. Polyclonal antibodies, including polyclonal antisera or polyclonal mixtures made by blending monoclonal and/or polyclonal antibodies with broad specificity across the different Gram-negative and Gram-positive species are useful in the devices and methods of the invention.

The antibodies indicated above can be utilized as described or modified as necessary to produce a useful immunological reagent.

In some embodiments, the particles useful in the binding assays and lateral flow device of the invention are one or more of gold, silver, or platinum particles. The particles can be of a uniform size, or they can be multiple sizes. In some embodiments, the particles can have a size of 10 nm to 150 nm, for example from 20 nm to 50 nm, from 40 nm to 80 nm, or from 60 nm to 100 nm. Exemplary particle sizes include 10 nm, 20 nm, 30 nm, 40 nm, 50 nm, 60 nm, 70 nm, 80 nm, 90 nm, 100 nm, 110 nm, 120 nm, 130 nm, 140 nm, and 150 nm. In certain embodiments, at least some of the particles are sized from about 60 nm to about 120 nm. In certain embodiments, all of the particles are sized from about 60 nm to about 120 nm. In certain embodiments, the particle size is about 80 nm. In other embodiments, the particle size is about 40 nm. In yet other embodiments, the device or method may comprise subpopulations of particles having different sizes, e.g., a subpopulation of 40 nm particles and a subpopulation of 80 nm particles. In certain embodiments, the device or method may comprise 40 nm particles and 80 nm particles, wherein a pan-generic monoclonal antibody is immobilized to the 40 nm particles and a different pan-generic polyclonal antibody is immobilized to the 80 nm particles.

In some embodiments, the pan-generic binding agent thereof is immobilized on the particle via a linker. In certain embodiments, the linker between a particle and a pan-generic binding agent is a protein linker (e.g., Protein L, Protein A, Protein G, or Protein A/G), or biotin-avidin, streptavidin, or neutravidin, or an anti-species antibody to immobilize another antibody on the conjugate (e.g., an anti-rabbit or anti-mouse antibody), agents capable of binding a recombinant protein tag (e.g., a His tag or a FLAG tag), DNA or a DNA-like molecule, or a synthetic immunoglobulin-binding moiety (e.g., a ProMetric BioSciences mimetic ligand).

In some embodiments, the device is a lateral flow device suitable for use in detecting bacteria in a blood sample or a blood product sample, the device comprising a flow path for the sample and a pan-generic binding agent (e.g., a pan-generic antibody) that binds a plurality of bacterial antigens, wherein the pan-generic binding agent is immobilized on a population of 80 nm gold particles, and further comprising a pan-generic binding agent (e.g., a pan-generic antibody) that is immobilized on a population of 40 nm gold particles. In further embodiments, the pan generic binding agents bind one or more Gram-positive bacterial antigens, one or more Gram-negative bacterial antigens, or both. In various embodiments, a pan-generic binding agent that binds a Gram-positive bacterial antigen may be on the same population or on a different population of gold particles (e.g., 80 nm gold particles) as a pan-generic binding agent that binds a Gram-negative bacterial antigen. In certain embodiments, a pan-generic binding agent immobilized on an 80 nm gold particle is a polyclonal antibody and a pan-generic binding agent immobilized on a 40 nm gold particle is a monoclonal antibody. In further embodiments, the device comprises a capture binding agent (e.g., a capture antibody) immobilized on the flow path of the device, wherein the gold particles are disposed along the flow path such that the sample contacts the population of colored particles before contacting the capture binding agent. In certain embodiments, the capture binding agent is a pan-generic binding agent. In embodiments in which the capture binding agent is a pan-generic binding agent, the capture binding agent may be the same as the pan-generic binding agent immobilized on the gold particles or may be different from the pan-generic binding agent immobilized on the gold particles.

### Methods of Detecting Bacteria

In some embodiments, the invention provides a device and method with broader reactivity than existing devices and methods. In particular, the devices and methods are capable of detecting a broader range of bacterial genera, species, and/or strains of bacteria than existing devices and methods. For example, the devices and methods may be capable of detecting at least 100, 150, 200, 250, 300, 350, 400, 450, or 500 different bacteria. In some embodiments, the invention provides a method or device comprising a pan-generic antibody capable of detecting greater than 1×10⁷, 1×10⁶, 1×10⁵, 1×10⁴, 1×10³, or 1×10² colony forming units (CFU) per mL of bacteria or an equivalent concentration of antigens derived from that level of bacteria.

In some embodiments, the invention provides a method to screen for the presence of bacteria in a liquid sample. In various embodiments of the method, the sample may be any biological fluid, including a dialysis sample. In some embodiments, the dialysis sample is selected from hemodialysis fluid and peritoneal dialysis fluid. In some embodiments, the sample is a sample of fluid in which a tissue has been stored. In some embodiments, the tissue is selected from the group consisting of blood cell cultures, stem cell cultures, and bone and cartilage graft materials. In some embodiments the sample is blood or a blood product including but not limited to whole blood, leukocytes, hematopoietic stem cells, platelets, red blood cells, plasma, bone marrow and dialysis fluid, comprising contacting a lateral flow device of the invention with a sample and detecting binding of the populations of antibodies to the sample, wherein binding indicates the presence of bacteria in the sample and no binding indicates the absence of bacteria in the sample. In certain embodiments, the sample is a dialysis fluid including hemodialysis fluid or peritoneal dialysis fluid.

In some embodiments, the invention provides a method to screen for the presence of bacteria in food or beverage products or food or beverage processing. For example, the methods of the invention could be used to test for the presence or absence of bacteria in lines used to carry liquids such beer or milk. The methods also could be used to test for the presence or absence of bacteria in water samples. In some embodiments, these methods comprise contacting a lateral flow device of the invention with a sample of a beverage or water sample and detecting binding of the populations of antibodies to the sample, wherein binding indicates the presence of bacteria in the beverage or water sample and no binding indicates the absence of bacteria in the beverage or water sample.

In certain embodiments of the invention, the sample is treated prior to or concomitantly with contacting the sample with a pan-generic antibody. Preferably, the treatment exposes a binding site for the pan-generic antibody on the Gram-negative bacterial antigen or on the Gram-positive bacterial antigen. A binding site on a bacterial antigen may be exposed by, for example, cleaving an antigen from the cell wall or cell membrane of the bacteria, thereby exposing the binding site; inducing the bacteria to secrete the antigen, thereby exposing the binding site; lysing the bacteria, thereby releasing an intracellular bacterial antigen and thus exposing the binding site on the antigen; or by inducing a conformational change on the bacterial antigen, thereby exposing the binding site. Such treatments include mechanical disruption of the bacterial cells in the sample by physical means, including, without limitation, sonication, boiling, or homogenization using, for example, a Dounce homogenizer. The treatment may also be treatment of the sample by chemical means with a compound or composition, such as detergent, a basic solution (for alkaline lysis), an acidic solution (for acidic lysis), EDTA, EGTA, a metal ion, an anion, a cation, a surfactant, a chelator, and/or an enzyme (e.g., lysostaphin, lysozyme, mutanolysin, labiase, achromopeptidase, trypsin, proteinase K, an autolysin, bacteriophage-encoded lytic enzymes, and combinations thereof). The treatment exposes a binding site for the pan-generic antibody on the Gram-negative bacterial antigen or on the Gram-positive bacterial antigen.

In some embodiments, the method is for use in detecting bacteria in a blood sample or a blood product sample, the method comprising contacting the sample with a pan-generic binding agent (e.g., a pan-generic antibody) that binds a plurality of bacterial antigens, wherein the pan-generic binding agent is immobilized on a population of 80 nm gold particles, and further comprising a pan-generic binding agent (e.g., a pan-generic antibody) that is immobilized on a population of 40 nm gold particles. In various embodiments, the method comprises contacting the sample with the pan-generic binding agent under conditions that permit binding between the pan-generic binding agent and the bacterial antigen and contacting an immobilized capture binding agent (e.g., a pan-generic binding agent such as a pan-generic antibody) with the gold particle under conditions that permit binding between the immobilized capture binding agent and the gold particle with the immobilized pan-generic binding agent. In certain embodiments, pan-generic binding agents bind one or more Gram-positive bacterial antigens, one or more Gram-negative bacterial antigens, or both. In various embodiments, a pan-generic binding agent that binds a Gram-positive bacterial antigen may be on the same population or on a different population of gold particles (e.g., 80 nm gold particles) as a pan-generic binding agent that binds a Gram-negative bacterial antigen. In certain embodiments, a pan-generic binding agent immobilized on an 80 nm gold particle is a polyclonal antibody and a pan-generic binding agent immobilized on a 40 nm gold particle is a monoclonal antibody. In further embodiments in which the capture binding agent is a pan-generic binding agent, the capture binding agent is the same as the pan-generic binding agent immobilized on the gold particles or is different from the pan-generic binding agent immobilized on the gold particles.

In a further aspect, the invention provides a kit comprising a detectable particle, such as a colored particle, including a gold, silver or platinum particle wherein the particle is sized about 60 nm to about 120 nm and wherein the particle comprises a multivalent binding agent immobilized thereon either directly or via a linker. In some embodiments, the multivalent binding agent is pan-generic binding agent such as a pan-generic antibody for the detection of Gram-negative bacteria, Gram-positive bacteria or both in a sample. In some embodiments, the particle is about 80 nm. In some embodiments, the kit comprises detectable particles of different sizes, such as 80 nm and 40 nm. In some embodiments, the kit comprises 80 nm gold particles with or without 40 nm gold particles. The kit further comprises instructions for using the detectable particle to detect the presence of bacteria in a sample. In some embodiments, the kit further comprises a solid surface having a capture pan-generic antibody immobilized thereon. In some embodiments, the solid surface is a component of a lateral flow device. In some embodiments, the kit further comprises a reagent for pretreating a sample.

The following examples are intended to further illustrate certain embodiments of the invention and are not intended to limit the scope of the invention.

### EXAMPLES

### Example 1

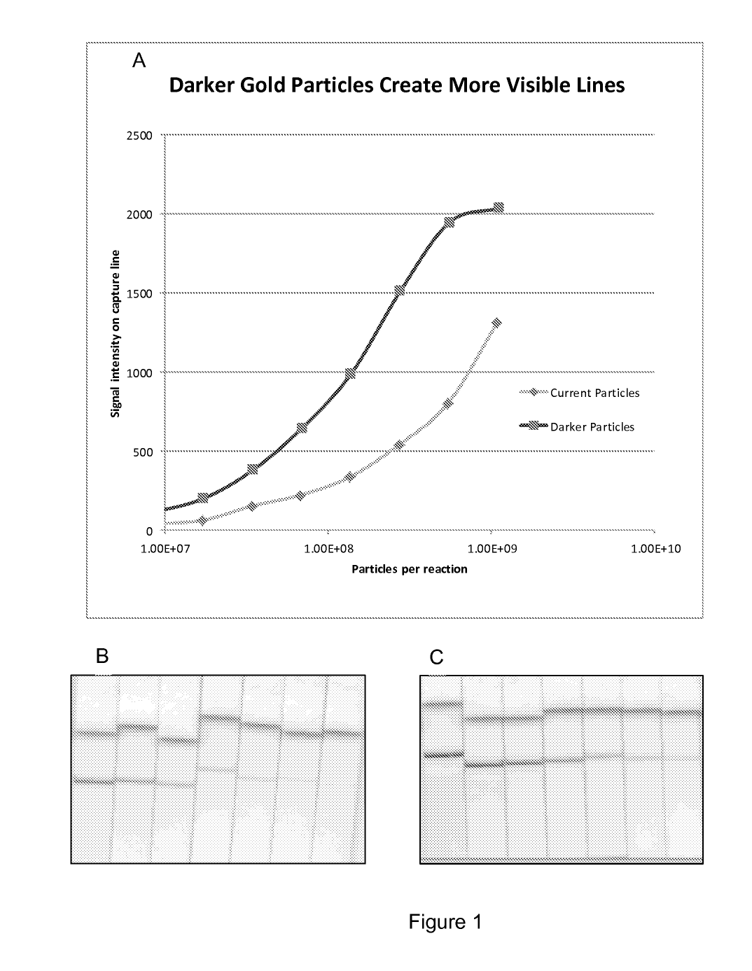

We measured the visual signal generated from different sized (40 nm and 80 nm) gold particles in a model lateral flow system to determine which particles gave the greatest signal intensity response per particle (FIGS. 1A-1C). The system was designed to capture a high proportion of the particles flowing through the strip, to give an indication of the visual signal produced by particles of varying sizes. A lateral flow device according to the invention was used. In this model the flow device utilized an IgG antibody striped on a Millipore nitrocellulose membrane as a capture binding agent, and protein A coated gold particles flowing through the strip. For a given number of particles added to the reaction. An 80 nm gold particle resulted in a higher contrast intensity purple line as compared to the red/pink line produced by the 40 nm gold particles, making the lines from the 80 nm beads easier to visualize and interpret. FIGS. 1B and 1C are images of the strips produced using varying numbers of 40 nm and 80 nm particles, respectively. The images were analyzed using Gelanalyzer 2010 software to provide values for the intensity of the capture lines. FIG. 1A shows a plot of signal intensity vs. the number of particles added to the reactions, demonstrating the increased signal intensity produced by equal numbers of larger gold particles.

Surprisingly, increasing the size of the particle also increases the intensity of the visually detectable signal generated on the capture line, thereby increasing the sensitivity. However, more numerous smaller particles, which have a larger surface to volume ratio than larger particles, would be expected to yield a better signal with faster results. Additionally, the amount of gold in the capture area is limiting, as a practical matter. Thus, it was particularly surprising that a lower amount of larger particles yielded better results than a higher amount of smaller particles.

### Example 2

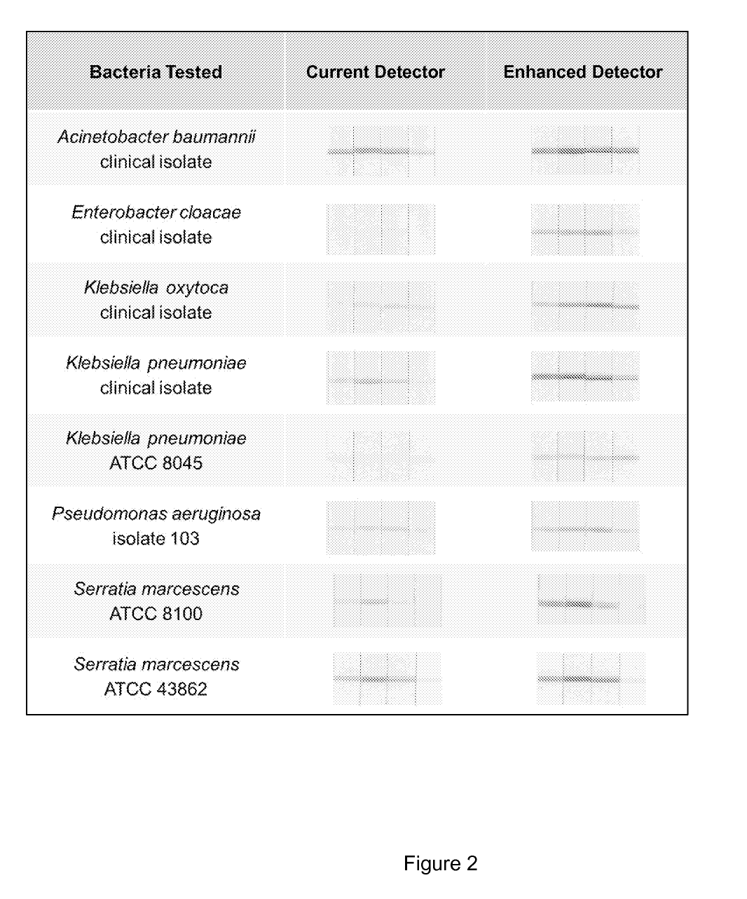

To test the effectiveness of the gold particles of different sizes, we constructed a model immunoassay system using a mixture of antibodies raised against a variety of Gram-negative and Gram-positive bacteria. We coupled the antibodies to 80 nm colloidal gold (“enhanced detector”) particles and compared their performance to 40 nm colloidal gold (“current detector”) particles. We prepared four levels of bacterial lysates for each of eight organisms by making tenfold dilutions starting at 10⁸ CFU/mL using a buffered solution. For each lysate level, we mixed 20 μL of current detector particles or enhanced detector particles (OD5), 20 μL of bacterial lysate, and 20 μL of a running buffer containing detergents in wells of a 96-well plate. A 0.5 cm dipstick cut from a Millipore nitrocellulose membrane card striped with the same antibody and laminated to an upper absorbent wick was inserted into each well and incubated until all of the liquid flowed into the dipstick. A chase of 100 μL PBS was used to clear the dipstick so it could be visually graded for signal intensity on a 1-12 scale vs. an intensity standard (deposited dilutions of particles) (Table 1 and FIG. 2).

TABLE 1
Signal intensity of current detector particles vs. enchanced detector
particles

| | Current Polyclonal | Enhanced Polyclonal |
|---|---|---|
| Bacteria Tested | Current Detector | Enhanced Detector |
| Acinetobacter baumannii | 10, 11, 10, 4 | 10, 11, 11, 10 |
| clinical isolate | | |
| Enterobacter cloacae | 2, 2, 3, 2 | 7, 8, 9, 5 |
| clinical isolate | | |
| Klebsiella oxytoca | 3, 4, 6, 4 | 5, 8, 10, 8 |
| clinical isolate | | |
| Klebsiella pneumoniae | 5, 8, 6, 2 | 10, 11, 11, 6 |
| clinical isolate | | |
| Klebsiella pneumoniae | 3, 3, 3, 3 | 5, 6, 6, 7 |
| ATCC 8045 | | |
| Pseudomonas aeruginosa | 6, 6, 7, 3 | 4, 8, 8, 3 |
| isolate 103 | | |
| Serratia marcescens | 3, 8, 2, 0 | 9, 10, 7, 1 |
| ATCC 8100 | | |
| Serratia marcescens | 9, 10, 9, 4 | 9, 11, 10, 4 |
| ATCC 43862 | | |

In all cases, the enhanced detectors were at least as sensitive as the current detectors, and in many cases, the signal was dramatically increased with the enhanced detector particles as compared to the current detector particles. For some bacterial species we observed sensitivity that was at least one log greater when using the enhanced detector particles as compared to the current detector particles.

In a multiple analyte system, increasing the size of the particle also increases the intensity of the signal generated in the antigen/antibody response, thereby increasing the sensitivity. This is surprising because the amount of gold in the capture area is limiting, as a practical matter. Thus, more numerous smaller particles can be used, and the smaller particles have a larger surface to volume ratio. Without wishing to be bound by theory, this phenomenon appears to occur because there are more antibodies per particle, thereby allowing greater avidity for an antigen. In these experiments, the gold particle, the immobilization method, and the conditions of binding the antibodies to the surface were evaluated. The result of these studies was successful immobilization of approximately four times more antibodies per gold particle, which yielded a substantially enhanced detector particle.

In summary, we have demonstrated increased signal intensity across multiple bacterial species by using larger, darker gold particles, resulting in improved sensitivity and accuracy with easier to read results.

### Example 3

### Synthesis of a Pan-Generic Reagent Particle

Rabbit IgG is diluted to desired concentrations in 2-fold concentrated binding buffer. Those of skill in the art will appreciate that, any binding buffer suitable for binding IgG to Protein A can be used. Typical concentrations for coupling range from 0.1 to 1 μg/ml*OD of gold. If 5 ml of gold colloid at OD555=10 is to be coupled with a ratio of 0.1 ug/ml*OD, then IgG is diluted to a concentration of 1.0 ug/ml in a volume of 5 ml. Diluted antibody is mixed with an equal volume of 80 nm gold particles coated with protein A (sPA) concentrated to twice the desired final desired concentration of particles. Incubation is for a minimum of one hour before testing, but overnight incubation also could be advantageous.

What is claimed is:

1. A lateral flow device for detecting bacteria in a sample, the device comprising a flow path for the sample and further comprising a pan-generic binding agent that specifically binds a bacterial antigen, wherein the pan-generic binding agent is immobilized on a population of particularly-sized colored particles; and a capture binding agent that captures the population of particles, wherein the capture binding agent is immobilized on the flow path, and wherein the population of detectable particles are disposed along the flow path such that the sample contacts the population of colored particles before contacting the capture binding agent.

2. The device according to claim 63, wherein the detectable particle is a colored particle selected from one or more of gold, silver and platinum particles.

3. The device according to claim 2, wherein the colored particle is a gold particle

4. The device according to claim 2, wherein the particle is from about 60 to about 120 nm in diameter.

5. The device according to claim 4, wherein the particle is about 80 nm in diameter.

6. The device according to claim 3, wherein the particle is from about 60 to about 120 nm in diameter.

7. The device according to claim 6, wherein the particle about 80 nm in diameter.

9. The device according to claim 1, wherein the binding agent is a pan-generic antibody.

10. The device according to claim 1, wherein the pan-generic binding agent specifically binds a Gram-positive bacterial antigen.

11. The device according to claim 10, wherein the pan-generic binding agent is a polyclonal antibody that binds lipoteichoic acid (LTA).

12. The device according to claim 1, wherein the pan-generic binding agent specifically binds a Gram-negative bacterial antigen.

13. The device according to claim 12, wherein the pan-generic binding agent is a pan-generic polyclonal antibody that binds a bacterial lipopolysaccharide structure (LPS).

14. The device according to claim 1, wherein at least one pan-generic binding agent specifically binds a Gram-positive bacterial antigen and at least one pan-generic binding agent specifically binds a Gram-negative bacterial antigen.

15. The device according to claim 1, wherein the pan-generic binding agent binds three or more genera of bacteria.

16. The device according to claim 1, wherein the pan-generic binding agent is immobilized on the colored particle via a linker.

17. The device according to claim 1, wherein the pan-generic binding agent comprises two or more pan-generic antibodies, wherein each pan-generic antibody specifically binds a bacterial antigen, wherein each pan-generic antibody is immobilized on a separate subpopulation of colored particles; and wherein at least one pan-generic antibody is immobilized on a population of particularly sized colored particles.

18. The device according to claim 9, wherein the pan-generic antibody is selected from one or more of a polyclonal antibody and a monoclonal antibody.

19. The device according to claim 18, wherein the pan-generic antibody is polyclonal and binds a plurality of bacterial antigens.

20. The device according to claim 18, wherein the pan-generic antibody is polyclonal and binds a plurality of Gram-positive bacterial antigens.

21. The device according to claim 18, wherein the pan-generic antibody is polyclonal and binds a plurality of Gram-negative bacterial antigens.

22. The device according to claim 17, wherein at least one pan-generic antibody is a monoclonal pan-generic antibody and at least one pan-generic antibody is a polyclonal pan-generic antibody.

23. The device according to claim 22, wherein the pan-generic antibody specifically binds a Gram-positive bacterial antigen.

24. The device according to claim 22, wherein the pan-generic antibody specifically binds a Gram-negative bacterial antigen.

25. The device according to claim 17, wherein at least one pan-generic antibody specifically binds a Gram-positive bacterial antigen and at least one pan-generic antibody specifically binds a Gram-negative bacterial antigen.

26. The device according to claim 17, wherein the pan-generic antibody binds three or more genera of bacteria.

27. The device according to claim 17, wherein the pan-generic antibody is immobilized on the colored particle via a linker.

28. The device according to claim 1, wherein the device comprises at least three pan-generic binding agents that specifically bind a Gram-positive bacterial antigen, each pan-generic binding agent immobilized on a separate subpopulation of colored particles; and at least three pan-generic binding agents that specifically bind a Gram-negative bacterial antigen, each pan-generic binding agent immobilized on a separate subpopulation of colored particles.

29. The device according to claim 28, wherein each subpopulation of particles is selected from one or more of gold, silver and platinum particles.

30. The device according to claim 29, wherein each subpopulation of particles is a gold particle.

31. The device according to claim 28, wherein at least one pan-generic binding agent is an antibody.

32. The device according to claim 31, wherein at least one pan-generic antibody is a monoclonal antibody.

33. The device according to claim 28, wherein the subpopulations of particles are of different sizes.

34. The device according to claim 33, wherein at least one gold particle population comprises a particle from about 60 nm to about 120 nm in diameter.

35. The device according to claim 34, wherein at least one gold particle population comprises an 80 nm gold particle.

36. The device according to claim 33, wherein at least one gold particle population comprises a 40 nm gold particle.

37. The device according to claim 1, wherein the capture binding agent is a pan-generic antibody that specifically binds a bacterial antigen.

38. The device according to claim 1, wherein the capture binding agent is the same as the pan-generic binding agent.

39. The device according to claim 37, wherein the capture binding agent is immobilized in two or more locations on the sample flow path.

40. The device according to claim 1, wherein the sample flow path is an absorbent membrane.

41. The device according to claim 40, wherein the absorbent membrane is nitrocellulose.

42. The device according to claim 1, wherein the colored particles are dried within a solid support surface disposed above the absorbent membrane and in contact with the upper surface of the membrane.

43. A method for detecting bacteria in a sample, comprising contacting the sample with a pan-generic binding agent specific for a bacterial antigen, wherein the pan-generic binding agent is immobilized on an particularly-sized colored particle, and wherein the sample is contacted with the pan-generic binding agent under conditions that permit binding between the pan-generic binding agent and the antigen, and further comprising contacting an immobilized capture binding agent with the colored particle under conditions that permit binding between the immobilized capture binding agent and the colored particle with the pan-generic binding agent, wherein capture of the colored particle with the pan-generic binding agent by the capture binding agent indicates the presence of bacteria in the sample.

44. The method according to claim 43, further comprising adding soluble pan-generic binding agent to the sample.

45. The method according to claim 43, wherein the method comprises contacting a device according to claim 1 with a sample under conditions that permit binding of the capture antibody to the colored particle with the pan-generic antibody, wherein capture of the particle by the capture antibody indicates the presence of bacteria in the sample.

46. The method according to claim 43, wherein the sample has been pre-treated.

47. The method according to claim 45, wherein the capture binding agent binds to the pan-generic antibody.

48. The method according to claim 43, wherein the sample is blood or a blood product.

49. The method according to claim 48, wherein the blood or blood product is selected from the group consisting of: whole blood, leukocytes, hematopoietic stem cells, platelets, red blood cells, plasma, bone marrow and serum.

50. The method according to claim 43, wherein the sample is a dialysis sample.

51. The method according to claim 42, wherein the dialysis sample is selected from hemodialysis fluid and peritoneal dialysis fluid.

52. The method according to claim 43, wherein the sample is a sample of fluid in which a tissue has been stored.

53. The method according to claim 44, wherein the tissue is selected from the group consisting of: blood cell cultures, stem cell cultures, bone and cartilage graft materials.

54. A reagent for use in a binding assay comprising a particle selected from a gold particle, a silver particle and a platinum particle, wherein the particle size is from about 60 nm to about 120 nm, and wherein the particle is bound to a multivalent binding agent.

55. The reagent according to claim 54, wherein the particle size is about 80 nm.

56. The reagent according to claim 54, wherein the pan-generic binding agent is bound to the particle via a linker.

57. The reagent according to claim 56, wherein the linker is selected from protein A, protein G and protein L.

58. The reagent according to claim 57, wherein the linker is protein A.

59. The reagent according to claim 54, wherein the particle is a gold particle.

60. The method according to claim 54, wherein the multivalent binding agent is a pan-generic binding agent.

61. A method for detecting a substance in a multianalyte sample comprising mixing the sample with a reagent according to claim 54, wherein binding of the substance to the reagent creates a detectable complex; and detecting the complex.

62. The method according to claim 59, wherein the method is an immunoassay.

63. The lateral flow device of claim 1, wherein the detectable particle is selected from chemiluminescent, a luminescent, a fluorescent, a magnetic or a colored particle.
